(19) United States
(12) Patent Application Publication
(10) Pub. No.: US 2021/0338279 A1
(43) Pub. Date: Nov. 4, 2021

(54) APPARATUS, SYSTEM AND METHOD FOR ACQUIRING A RECORDING FROM WITHIN A SUBJECT

(71) Applicant: NEURAURA BIOTECH INC., Calgary (CA)

(72) Inventors: Pierre Jean Jacques WIJDENES, Calgary (CA); Colin DALTON, Calgary (CA); Syed Kazim HAIDER, Calgary (CA)

(21) Appl. No.: 17/282,231

(22) PCT Filed: Nov. 8, 2019
(86) PCT No.: PCT/CA2019/051597

§ 371 Date: Apr. 1, 2021

Related U.S. Application Data
(60) Provisional application No. 62/758,302, filed on Nov. 9, 2018.

(51) Int. Cl. A61B 17/34; A61B 5/293

(52) U.S. Cl. CPC A61B 17/3468; A61B 2562/0209; A61B 2560/063; A61B 5/293

(57) ABSTRACT

The present disclosure relates to an apparatus, system and method for inserting one or more probes into a subjects body for one or more purposes of acquiring physiological data or delivering a treatment into the subjects body.

### TECHNICAL FIELD

This disclosure generally relates to acquiring data from a subject. In particular, the disclosure relates to an apparatus, system and method for acquiring a recording, such as a physiological recording, from within the subject.

### BACKGROUND

Acquiring data, such as physiological data, from a subject is important to assess the subject's health. This data can provide an assessment of one or more of the subject's physiological systems and/or one or more pathophysiological processes that may be occurring within the subject. Furthermore, based upon the acquired data, it may be desirable to deliver a treatment to the subject that is targeted to a specific physiological system and/or specific tissue or portion of that system.

Within the central nervous system and, in particular, the brain it can be challenging to acquire data and to deliver targeted therapies due to the skull and other layers of tissue that protect the brain.

### SUMMARY

Some embodiments of the present disclosure relate to a guide for inserting one or more probes into a subject for collecting physiological data therefrom and/or to deliver a treatment thereto. The guide may comprise an elongate body with a first end and a second end. The elongate body defines a slot that extends between the first end and the second end. The slot is configured to receive a first probe at the first end and to define an angle, relative to the elongate body, at which the first probe is extendible from the second end. In some embodiments of the present disclosure, there may be more than one slot each of which is configured to receive a probe and to define such an angle relative to the elongate body. In some embodiments of the present disclosure, the one or more slots are each configured to receive one or more probes.

Some embodiments of the present disclosure relate to a system for acquiring a neurocortical and/or subcortical recording. The system comprises a guide for inserting one or more probes into a subject's body, for example, within the subject's skull. The probes can be either a neurocortical electrode, a subcortical electrode or other types of tools that may be used to acquire physiologic data and/or deliver a treatment within the subject's body. The guide comprises an elongate body with a first end and a second end and the elongate body defines one or more slots between the first end and the second end. The slots are each configured to receive one or more probes at the first end and to define an angle relative to the slot at which the one or more probes are extendible from the second end. In some embodiments of the present disclosure, the probes may each be a neurocortical probe or a subcortical probe that are configured, respectively, to receive neurocortical signals or subcortical signals when positioned near, upon, or inside a portion of a subject's tissue, for example, neural tissue.

Some embodiments of the present disclosure relate to a method of introducing one or more probes into a subject's body. The method comprises the steps of accessing an interior of the subject's body via an access hole; inserting a guide that comprises an elongate body with a first end and a second end. The elongate body defines one or more slots that extend between the first end and the second end. The slots are each configured to receive one or more probes at the first end and to define an angle relative to the slot at which the probes are extendible from the second end. The method further comprises a step inserting the probes into the slots so that a second end of each probe extends from the elongate body at the desired angle for positioning the second end of each the probes near, upon, or inside a desired tissue structure within the subject's body.

Without being bound by any particular theory, the embodiments of the present disclosure may allow for the acquisition of neurocortical signals from the surface of the subject's brain without requiring large portions of the subject's skull and cerebral-protective tissue to be removed. Instead, a hole can be drilled into the subject's skull and the guide can be secured to a portion of the subject's skull and the guide can be used to introduce one or more probes near, upon, or within the subject's brain. In some embodiments of the present disclosure, at least one or more probes may be a neurocortical electrode and positioning of the neurocortical electrode may allow for acquiring of neurocortical signals. Neurocortical signals may provide a more complete view of the neurophysiology in larger portions of the subject's neurocortex. This is in contrast with elongate-shaped electrodes, which may also be referred to as depth electrodes, deep electrodes, or subcortical electrodes, which extend deeper into the subject's brain, below the surface, to acquire signals from deeper and/or more localized regions of the subject's brain. Furthermore, in some embodiments of the present disclosure, the guide may also define one or more secondary slots each for inserting one or more tools to deliver a treatment within the subject's skull. In some embodiments of the present disclosure, it may be possible to acquire neurocortical and/or subcortical signals from the subject's brain while the one or more tools are being used to perform one or more neurosurgical procedures on the subject's brain, such as: therapy delivery, treatment delivery, acquiring further physiologic data within a localized portion of the subject's brain or combinations thereof.

### BRIEF DESCRIPTION OF THE DRAWINGS

These and other features of the present disclosure will become more apparent in the following detailed description in which reference is made to the appended drawings.

FIG. 1 provides a number of views of a guide according to a first embodiment of the present disclosure, wherein: FIG. 1A is an isometric view of the first embodiment showing a second end of the guide; FIG. 1B is another isometric view of the first embodiment showing a first end of the guide; FIG. 1C is a partial cut-away, isometric view of the first embodiment; FIG. 1D is a side-elevation view of the first embodiment; FIG. 1E is a partial cut-away, side-elevation view of the first embodiment; FIG. 1F is a mid-line, cross-sectional view of an optional variation of the first embodiment.

FIG. 2 shows an embodiment of a guide and sealing apparatus, wherein FIG. 2A shows a side elevation, exploded view, FIG. 2B shows a closer view of two components shown in FIG. 2A; and, FIG. 2C shows a side elevation, partial exploded view.

FIG. 3 shows another embodiment of a guide and sealing apparatus.

FIG. 4 shows another embodiment of a guide and sealing apparatus, wherein FIG. 4A shows a side elevation, exploded view; and, FIG. 4B shows a side elevation, partially exploded view.

FIG. 5 provides a number of views of a guide according to a second embodiment of the present disclosure, wherein: FIG. 5A is an isometric view of the second embodiment showing a second end of the guide; FIG. 5B is another isometric view of the second embodiment showing a first end of the guide; FIG. 5C is a side-elevation view of the second embodiment; FIG. 5D is a partial cut-away, side-elevation view of the second embodiment; FIG. 5E is a partial cut-away, side-elevation view of an optional variation of the second embodiment.

FIG. 6 provides a number of views of a guide according to a third embodiment of the present disclosure, wherein: FIG. 6A is a side-elevation view of the third embodiment; FIG. 6B is a closer isometric view of a second end of the third embodiment taken from circle X shown in FIG. 6A and showing the second end of the guide; FIG. 6C is an isometric view of the third embodiment showing a first end of the guide; FIG. 6D is a partial cut-away, side-elevation view of the third embodiment; and FIG. 6E is a partial cut-away, side-elevation view of an optional variation of the third embodiment.

FIG. 7 provides a number of views of a guide according to a fourth embodiment of the present disclosure, wherein: FIG. 7A is an isometric view of the fourth embodiment that shows a second end of the guide; FIG. 7B is another isometric view of the fourth embodiment that shows a first end of the guide; FIG. 7C is a side-elevation view of the fourth embodiment; FIG. 7D is a closer isometric view of the second end of the fourth embodiment taken from circle Y shown in FIG. 7C; FIG. 7E is a partial cut-away, side-elevation view of the fourth embodiment; and FIG. 7F is a closer isometric view of the second end of the fourth embodiment taken from circle Y′ shown in FIG. 7E.

FIG. 8 shows various views of a portion of a system according to the present disclosure that includes a guide and one or more neurocortical electrodes for use on a subject, wherein FIG. 8A is a front-elevation view of the subject; FIG. 8B is a rear-elevation view of the subject; FIG. 8C is a top-plan view of the subject; and FIG. 8D is a top-plan view of the subject with multiple guides and different, non-limiting combinations of neurocortical electrodes.

FIG. 9 shows different embodiments of a portion of a system according to embodiments of the present disclosure that includes a guide and one or more neurocortical electrodes for use on a subject.

### DETAILED DESCRIPTION

Embodiments of the present disclosure relate to an apparatus, system and method for acquiring one or more records of physiological data from within a subject, for delivering a treatment to a tissue structure within the subject's body, and/or for performing a procedure within the subject's body. In some embodiments of the present disclosure the physiological data may include thermal data, chemical data, electrical data, electrochemical data, anatomical data or combinations thereof. In some embodiments of the present disclosure, the physiological data relates to the subject's skeletomuscular system, immune system, lymphatic system, circulatory system, endocrine system, gastrointestinal system, nervous system, respiratory system or combinations thereof. In some embodiments of the present disclosure, the physiological data is electrochemical data that is acquired from the subject's nervous system, including the subject's peripheral nervous system and central nervous system.

Some embodiments of the present disclosure relate to an apparatus that can be used as a guide to introduce at least one probe into the subject's body without requiring that extensive sections of the subject's tissue be removed to provide access to the desired tissue structures and/or systems within the subject's body. While the present description discusses various non-limiting embodiments that relate to introducing a neurocortical probe and/or a subcortical probe and/or tool into a subject's skull for positioning the probes and/or tools near, upon, or within a portion of the subject's brain, the person skilled in the art will appreciate that the embodiments of the present disclosure may be equally useful for introducing different types of electrodes, sensors, tools, and therapies into a subject's body in a manner that is less invasive than other known approaches. For example, the apparatus may also be used to deliver various types of therapies and treatments to a desired tissue structure or system within a subject, including but not limited to: electrical stimulation; a chemical treatment, via a micro tube; a mechanical treatment, such as insertion of a stent or other medical device; delivery of a fiber optic cable for laser ablation; a thermal treatment, via a conductive wire; or combinations thereof. In some embodiments of the present disclosure, the apparatus may be used to guide two probes into a subject's body, where one probe comprises a sensory component (such as an electrode) and another probe comprises a treatment component (such as an optical fibre). In these applications, the guide can facilitate both recording of physiologic data and delivery of a therapy within the subject's body, either concurrently or not.

In some embodiments of the present disclosure, the guide may be inserted into a drilled hole in the subject's skull to introduce a neurocortical electrode inside the subject's skull and to position the neurocortical electrode near or upon a portion of the surface of the subject's brain. Neurocortical electrodes may have one or more individual electrodes that are planar in shape, or what is referred to as a three-dimensional electrode shape. Neurocortical electrodes with multiple individual electrodes connected on a planar substrate can form an array or strip of electrodes. When the planar substrate is positioned near or upon a portion of the surface of the subject's brain, the individual electrodes can detect electrical activity within a portion of the subject's brain. This example application of the embodiments of the present disclosure may avoid having to perform a craniotomy that includes forming a bone flap, removing the bone flap and then positioning the bone flap back in place to cover up the exposed region of the subject's brain. These types of craniotomies are typically necessary to introduce one or more probes and to position them near, upon or within a portion of the subject's brain. This example application is in contrast with other types of neuro-electrodes, such as depth electrodes, or subcortical electrodes, that can be introduced through the subject's skull and into the subject's brain by a drilled hole. These types of depth electrodes are typically more elongate shaped, rather than planar, and they are designed to penetrate into the brain so they can detect electrical signals within the brain rather than on the surface of the brain.

Definitions

Unless defined otherwise, all technical and scientific terms used herein have the same meaning as commonly understood by one of ordinary skill in the art to which this disclosure belongs.

As used herein, the term “about” refers to an approximately +/−10% variation from a given value. It is to be understood that such a variation is always included in any given value provided herein, whether or not it is specifically referred to.

As used here, the term “electrical communication” refers to a one-way flow of an electrical signal and/or a two-way exchange of electrical signals. The one-way flow of the electrical signal may originate in: at least one electrically-excitable cell, an electrode or both. The two-way exchange of electrical signals refers to both the transmission and receipt of electrical signals by an electrode and at least one electrically-excitable cell. Electrical communication may also refer to the detection and/or transmission of an electrical signal between the electrode and at least one electrically-excitable cell.

As used herein, the term “electrode” refers to an electrical conductor used to make contact with a non-metallic part of the circuit, and would include for example but is not limited to a 3-dimensional micro or macro-electrode, a planar micro or macro-electrode, an elongate micro or macro-electrode, a planar array of multiple micro or macro-electrodes, a planar array of multiple 3D micro or macro-electrodes.

As used herein, the term “electrically-excitable cell” refers to a cell that have the potential to communicate charged ions across the cellular membrane in response to an electric, chemical or physical stimuli. In some instances, the electrically-excitable cells can depolarize in a regulated fashion to generate and propagate one or more action potentials or end-plate potentials. Some examples of electrically-excitable cells include but are not limited to all types of neural cells, cardiac cells and muscle cells.

As used herein, the term “excitable cellular-network” and the acronym “ECN” refer to a network of electrically-excitable cells that are interconnected so that stimulation of one or more than one electrically-excitable cell within the network will elicit a response in at least one other electrically-excitable cell within the network. The response of at least one other electrically-excitable cell will include a communication of charged ions across its cellular membrane and/or the release of one or more extra-cellular signal molecules.

As used herein, the term “neurocortical” refers to upon or near the surface of the subject's brain cortex tissue. “Neurocortical signals” are a time series of acquired voltage signal measurements that correspond to neural activity upon or near the cortex tissue.

As used herein, the term “physiological data” refers to raw numerical measurements (i.e. numbers) that are proportional to the subject's homeostatic processes that are being acquired. Some examples of physiological data include electrical signals, neurocortical signals, subcortical signals, chemical signals, hormonal signals, cardiac signals, respiratory signals and the like.

As used herein, the term “physiological information” refers to information that is garnered through interpreting the acquired physiological-data and from which a skilled person may draw conclusions about recommended course of action, diagnosis, prognosis, treatment, further data capturing or combinations thereof.

As used herein, the term “probe” refers to an electrode or a surgical tool that is configured to acquire one or more sets of physiological data from the subject's body, such surgical tools may be configured to deliver one or more therapies or treatments, or such surgical tools may be a sensor, an oxygen probe, a temperature probe, a chemically-sensitive probe, an optical fiber, a stent insertion assembly, a stent, a neuro-medical device insertion assembly, a neuro-medical device, other tools that may be desired to introduce and position objects within a subject's body through the embodiments of the present disclosure.

As used herein, the term “slot” refers to a 3-dimensional geometric feature comprising a geometric profile defined in two-dimensions that extends a distance in the third-dimension such that there is a high aspect ratio between the distance extended by the profile in the third-dimension and the two measurable dimensions of the geometric profile. For example, a slot may be defined in the X-Y plane to have a rectangular, square, curvilinear, or circular profile, and this profile may extend a distance in the Z-dimension such that the boundaries of the slot's profile define a channel having a length equal to the distance extended in the Z-dimension. Similarly, a slot may be defined in any combination of the three-dimensions, for example having a profile defined in the X-Z plane and extending in the Y-dimension, and any combinations thereof.

As used herein, the term “subcortical” refers to below the surface of the subject's brain cortex tissue. “Subcortical signals” are a time series of acquired voltage signal measurements that correspond to neural activity below the cortex tissue and deeper within the subject's brain.

As used herein, the term “subject” refers to any therapeutic target that can receives the guide. The subject can be a vertebrate, for example, a mammal including a human. The term “subject” does not denote any particular age or gender.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or may be a therapy that provides a partial or complete amelioration or inhibition of a disease, disorder, or symptom thereof. Additionally, the term “treatment” refers to any treatment of a disease, disorder, or symptom thereof in a subject and includes: (a) inhibiting the disease, i.e., arresting its development; and (b) ameliorating the disease.

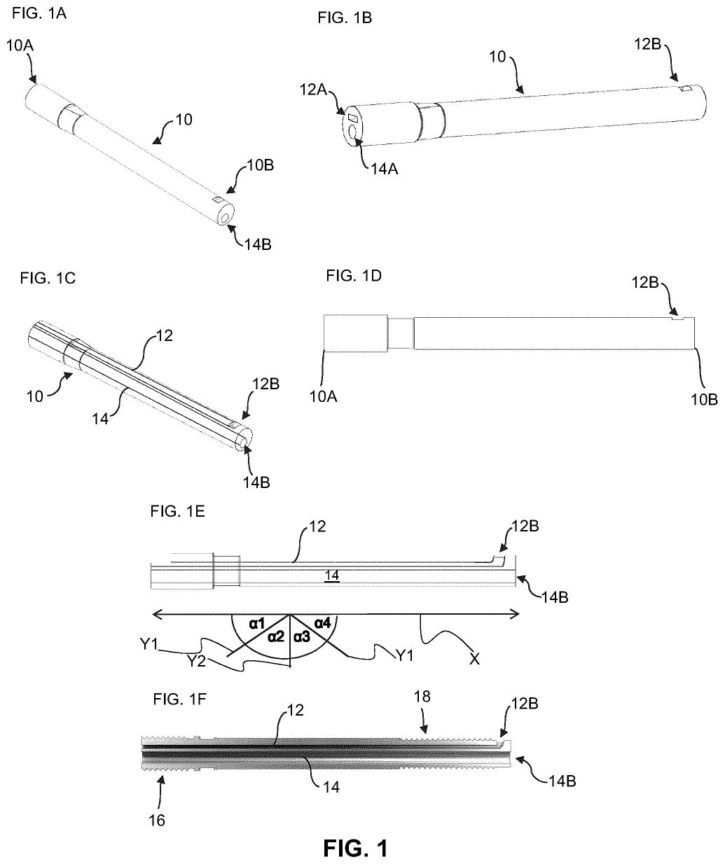

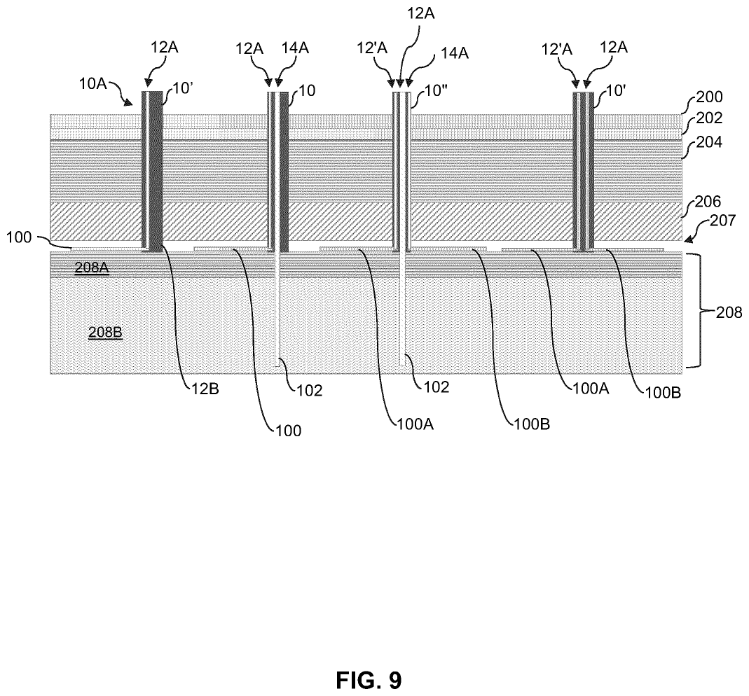

Embodiments of the present disclosure will now be described by reference to FIG. 1 through to FIG. 9, which show representations of apparatus, systems and methods according to the present disclosure that may be useful for introducing a probe into a subject's skull and for positioning the probe near or on a portion of a surface of the subject's brain.

FIG. 1A, FIG. 1B, FIG. 1C, FIG. 1D and FIG. 1E all show a first embodiment of an apparatus, referred to herein as a guide, that can be used to introduce a probe into a subject's skull and position the probe near or on a portion of a surface of the subject's brain.

The guide comprises a elongate body 10 that has a first end 10A and a second end 10B and a longitudinal axis X is defined therebetween. The elongate body 10 defines one or more slots 12 that each has a central axis that is substantially parallel to the longitudinal axis X of the elongate body 10. The slot 12 has a first end 12A defined proximal the first end 10A and a second end 12B defined proximal the second end 10B. The first end 12A of the slot 12 is configured to receive a probe therethrough. In some embodiments of the present disclosure, the probe may be an array of neuro-cortical electrodes. The second end 12B of the slot 12 is configured to define a desired angle relative to the longitudinal axis X at which the probe may exit and extend away from the elongate body 10. For clarity, towards first end 10A can represents an angle of 0 degrees relative to the longitudinal axis X of the elongate body 10 and towards the second end 10B represents an angle of 180 degrees relative to the longitudinal axis X of the elongate body 10.

As shown in FIG. 1E, the second end 12B of the slot 12 may be radiussed so that as the probe moves in a first direction from the first end 12A towards the second end 12B, when the probe reaches the radiussed section of the second end 12B the probe, which may be flexible, may bend and exit the slot 12 and extend away from the elongate body 10 at the desired angle relative to the longitudinal axis of the elongate body 10. FIG. 1E shows three lines Y1, Y2 and Y3 extending away from the longitudinal axis with four angles defined therebetween α1, α2, α3 and α4. Angle α1 may between about 1 degree and about 45 degrees relative to the longitudinal axis X.

The sum of angles α1 and α2 is between about 45 degrees and about 90 degrees relative to the longitudinal axis X. The sum of angles α1, α2 and α3 is greater than about 90 degrees but less than about 135 degrees relative to the longitudinal axis X. The sum of angles α1, α2, α3 and a4 is between about 135 degrees and about 180 degrees relative to the longitudinal axis X. In some embodiments of the present disclosure, the desired angle is between about 1 degree and about 179 degrees relative to the longitudinal axis of the elongate body. In some embodiments of the present disclosure, the desired angle may be between about 5 degrees and about 150 degrees relative to the longitudinal axis of the elongate body 10; between about 15 degrees and about 125 degrees relative to the longitudinal axis of the elongate body 10; or the desired angle may be greater than 90 degrees relative to the longitudinal axis of the elongate body 10. In some embodiments of the present disclosure, the predetermined angle can be modified by design of the radiussed portion of the second end 12B and/or by the selection of materials that the probe is constructed of.

In some embodiments of the present disclosure, the slot 12 may have a cross-sectional shape that generally corresponds to the cross-sectional shape of the probe. In some embodiments of the present disclosure, the slot 12 has a cross-sectional shape selected from one of a rectangular shape (as shown in FIG. 1), a square shape, a circular shape or a curvilinear shape.

In some embodiments, the probe can be a tool that is configured to acquire one or more sets of physiological data, such as thermal data, chemical data, electrical data, electrochemical data, anatomical data or combinations thereof. As such, the desired angle at which the electrode extends away from the elongate body, in conjunction with the depth at which the second end 10B is positioned within the subject may allow for acquisition of one or more points and/or sets of physiologically relevant data from within the subject's body. In some embodiments of the present disclosure, the tool may be a neurosurgical tool that is configured to deliver one or more treatment within the subject at, near or beyond the depth at which the second end 10B is positioned within the subject. In some embodiments, the probe can be a tool that is configured to facilitate the insertion of subsequent probes, such as a probe that delivers a pressurized stream of a gas or liquid, like air or saline, to create a gap between internal tissues such that subsequent probes can be slid through the gap created. In some embodiments, the first probe can also be used to introduce chemicals, like therapeutic drugs, or to deliver electrical signals or to generate electrical signals within the subject.

In some embodiments of the present disclosure, the first probe may comprise one or more of an array of neurocortical electrodes, a tool that is configured to acquire physiological data or a tool that is configured to deliver one or more treatments.

In the embodiment of a guide depicted in FIG. 1, the elongate body 10 also defines a secondary slot 14 that may be parallel with the longitudinal axis X of the elongate body and the slot 12. The secondary slot 14 has a first end 14A that is defined proximal the first end 10A and a second end 14B that is defined proximal to the second end 10B. The first end 14A of the secondary slot 14 may be configured to receive a further probe therein so that that the further probe can move through the secondary slot 14 in a first direction from the first end 14A towards the second end 14B and beyond the second end 14B so the further probe can continue moving in the first direction and extend away from the elongate body 10.

In some embodiments of the present disclosure, the probe received by the secondary slot 14 can be a tool that is configured to acquire physiological data, including but not limited to a depth electrode, or a subcortical electrode, or a tool that is configured to deliver one or more therapies or treatments but that does not require any angular deflection upon exiting the secondary slot 14.

FIG. 1F shows an optional variation of the first embodiment of the guide. In this variation the first end 10A comprises a first connection region 16 and/or a second connection region 18. The connection regions 16, 18 may be configured to secure the elongate body 10 in a semi-fixed or fixed position relative to another structure, such as a stereotactic apparatus or an anatomical structure of the subject. While the connections regions 16, 18 are shown as comprising threading, the person skilled in the art will appreciate that the connection members 16, 18 can comprise various different types of connections to secure the elongate body 10 in a desired location on or about the subject's body. The connection region 16 may also be configured to allow the attachment of different types of components external to the subject, such as a sealing apparatus, antennas, a passive thermal management solution, and the like.

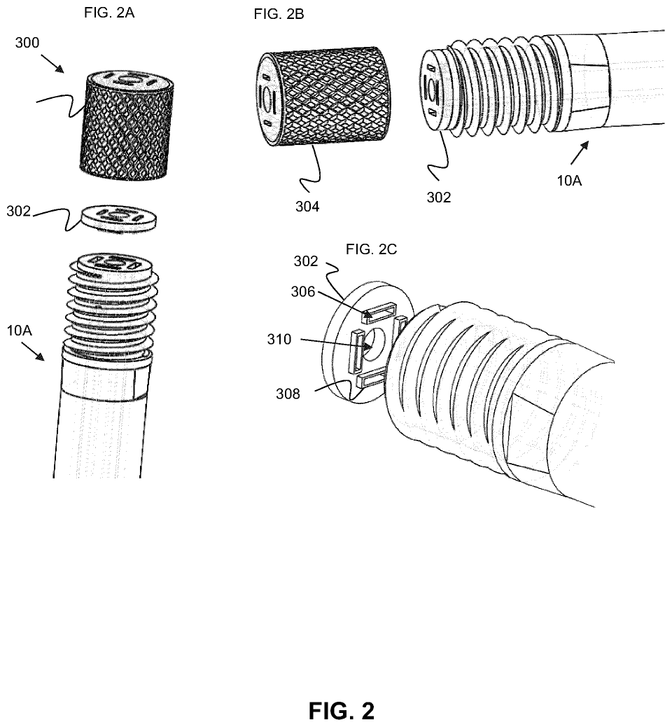

FIG. 2A shows another embodiment of the present disclosure that relates to a sealing apparatus 300 for use with the guide. The sealing apparatus 300 comprises a sealing plate 302 and a cap 304. The sealing plate 302 is configured to be reversibly connectible to the first end 10A (as shown in FIG. 2B) of the guide. The sealing apparatus 300 is also configured to provide a fluid tight seal about the first end to reduce, substantially prevent or stop relative motion between probes positioned within the guide and to reduce, substantially prevent or stop any leaking of the subject's bodily fluids from the second end 10B through to the first end 10A.

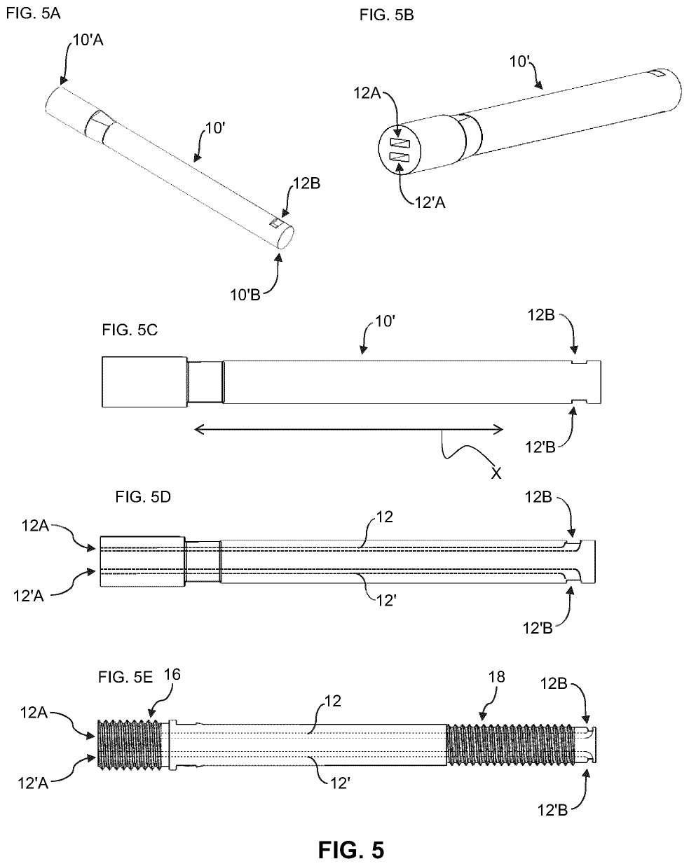

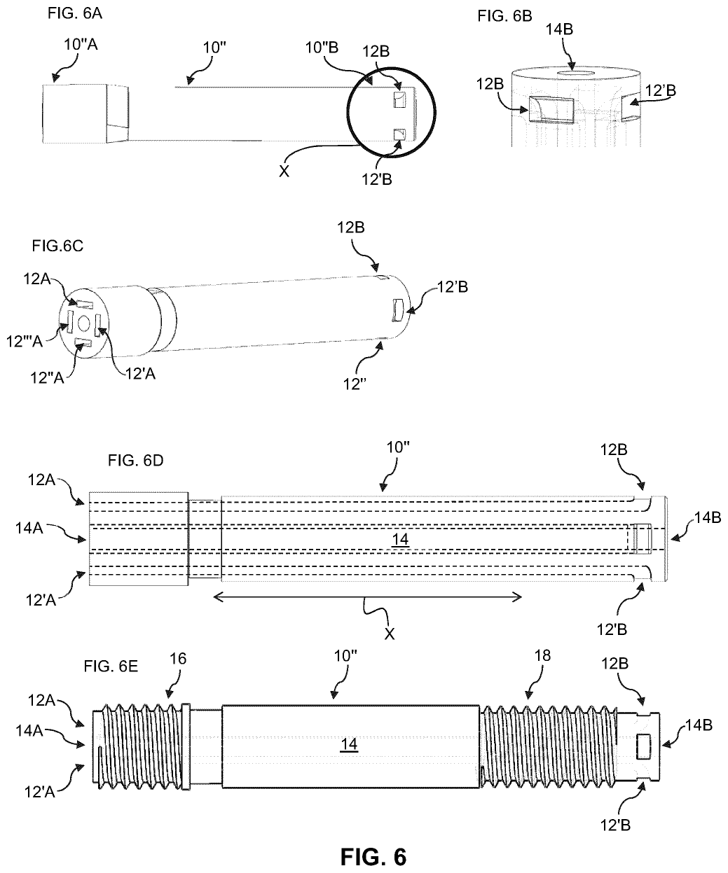

As shown in FIG. 2C, the sealing plate 302 may define one or more apertures 306 that are configured to substantially match the geometry of the slot 12. As will be appreciated by those skilled in the art, the one or more apertures 306 are configured to also substantially match the geometry and number of slots defined by the guide (for example see the slots 12A, 12′A, 12″A, 12′″A shown in FIG. 5 and FIG. 6) on the first end 10A so that a probe may be received through each aperture 306 and within the guide. The sealing plate 302 may further comprise one or more aperture extensions 308 that are each configured to be insertable into the slots 12A, 12′A, 12″A, 12′″A channel openings and securable therein by an interference fit. In some embodiments of the present disclosure, the cap 304 may further define a second aperture 310 that is configured to align with the secondary slot 14. The cap 304 may be reversibly connectible to the first end 10A and when so connected a substantially fluid-tight seal is established within the sealing apparatus 300 about the first end 10A that reduces leakage of fluids out of the first end 10A. Some examples of sealing mechanisms that may be contained inside the sealing apparatus 300 include, but are not limited to a gasket, a flexible sealing member, an o-ring or combinations thereof that are positioned at one or more of about an outer edge of the cap 304, within an inner surface of the one or more apertures 306, an outer surface of the one or more aperture extensions 308 and an inner surface of the second aperture 310. As will be appreciated by one skilled in the art, there are various types of sealing mechanisms, flexible or not, and various positions of said sealing mechanisms that will achieve the sealing function to prevent leaking.

In some embodiments of the present disclosure, the sealing plate 302 may define one or more apertures 306 but no aperture extensions 308 for example for probes that can be used with different types of sealing mechanisms. One example of a probe with an alternate sealing mechanism may be a depth electrode, which may have a sealing mechanism may be contained inside the cap 304. In some embodiments of the present disclosure, the cap 304 may have an external finish, like the knurled finish as shown in FIG. 2, to facilitate gripping and handling. The cap 306 may also comprise external markings such as arrows or letters that communicate information such as, but not limited to: alignment markings, a number and direction of turns to form a secure seal and the like.

In some embodiments of the present disclosure, the cap 304 may have threads (internal or external) that are configured to match threads (external or internal) on the first end 10A so that the cap 304 may be threadably and reversibly connected to the first end 10A.

The individual components of the sealing apparatus 300 may be made of the same materials or different materials that are suitable for positioning upon or inside the subject's body. In some embodiments of the present disclosure, the cap 304 may be made of, but is not limited to, titanium, titanium alloy, stainless steel, or a polymer, while the sealing plate 302 may be made of, but is not limited to, a flexible material like silicone, rubber, or a flexible or deformable metal or metal alloy.

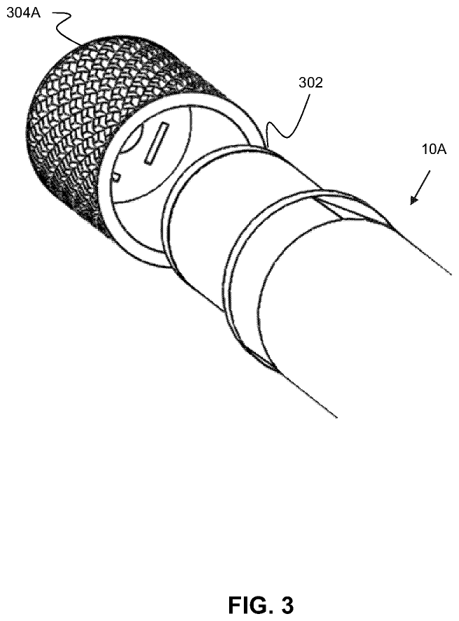

In another embodiment of the present disclosure, the sealing apparatus 300 comprises a cap 304A that is reversibly connectible to the first end 10A by a press-fit or interference fit that substantially prevents or stops relative motion of the probes, the sealing apparatus 300 and the guide (see FIG. 3).

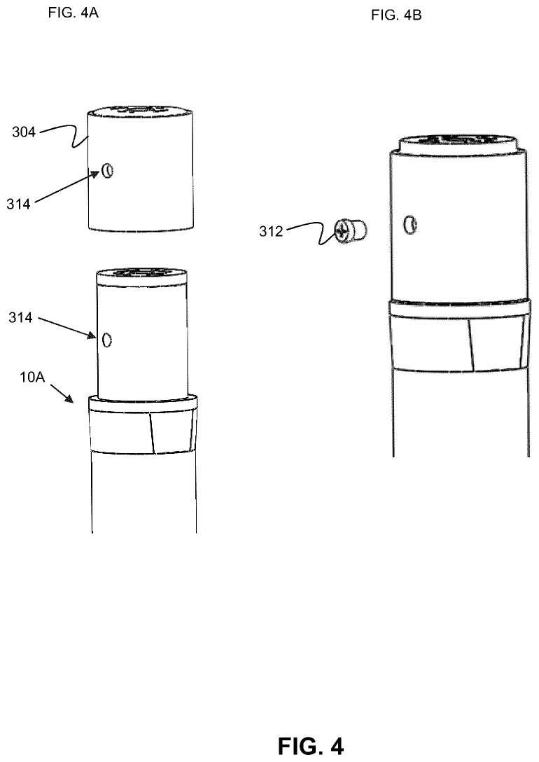

In another embodiment of the present disclosure, the sealing apparatus 300 comprises a cap 304B that is configured to be reversibly connectible to the first end 10A by one or more connection members 312. The connection member 312 may be a set screw, a grub screw, a blind screw, or it may have a protruding head (as shown in FIG. 4). The cap 304B may define one or more through-holes 314 with a thread that matches the thread of the connection member 312, or not. The guide may also define a matching threaded through-hole 314A that is also configured to receive the connection member 312. The depth of the through holes 314, 314A are such that the grub screw does not collide with any probes positioned within, at or near the first end 10A. The location and arrangements of the holes 314, 314A about the circumferential and axial directions of the cap 304B and the guide may vary based on the overall dimensions of the guide. The connection members 312 can be made of the same or different materials as the cap 304B.

As will be appreciated by those skilled in the art, the sealing apparatus 300 can be reversibly connected together and to the guide by various other mechanisms, such as a click arrangement, a ball and detent arrangement, a deformable and/resilient arrangement or combinations thereof.

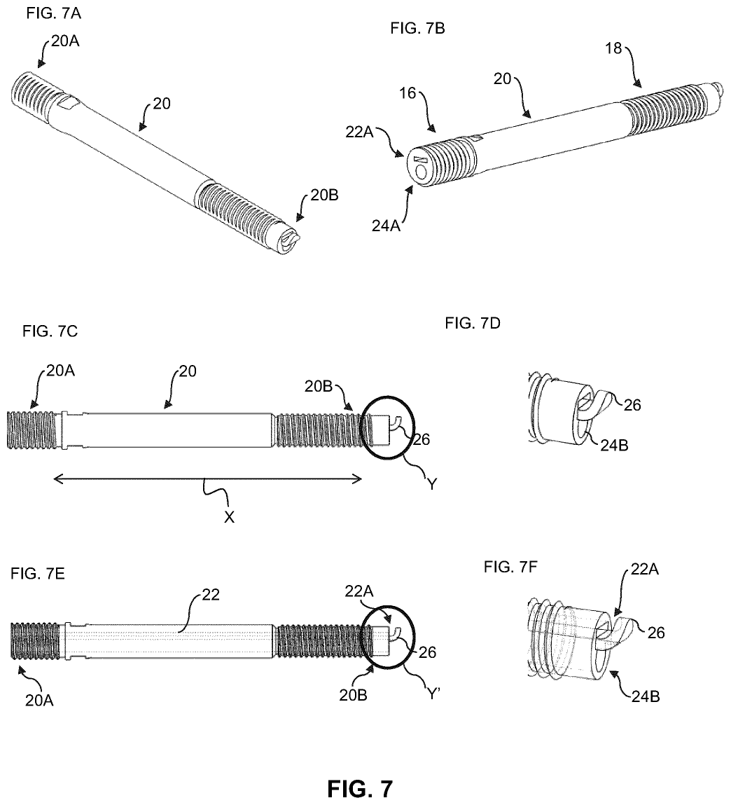

FIG. 5, FIG. 6 and FIG. 7 all show further, non-limiting embodiments of the guide. As will be appreciated by one skilled in the, the sealing apparatus 300 described above may be used with these further, non-limiting, embodiments of the guide.

FIG. 5A, FIG. 5B, FIG. 5C and FIG. 5D show a second embodiment of the guide. The second embodiment of the guide comprises many of the same features as the first embodiment of the guide described above. The guide comprises a elongate body 10′, a slot 12 and a second slot 12′. The elongate body 10′ comprises a first end 10′A and a second end 10′B and a longitudinal axis X extends therebetween. At least one difference between the first embodiment and the second embodiment is that the elongate body 10′ does not define the secondary slot 14 of the first embodiment. The slot 12 is as described herein above and, similarly, the second slot 12′ has a first end 12′A that is defined proximal the first end 10′A and a second end 12′B that is defined proximal to the second end 10′B. The second end 12′B of the second slot 12′ is, similar to the slot 12, configured to define a desired angle relative to the longitudinal axis X at which the electrode within the second slot 12′ may exit and extend away from the elongate body 10. The desired angle defined by the second end 12′B may be the same as the desired angle defined by the second 12B of the slot 12, or not.

FIG. 5E shows an optional variation of the second embodiment that includes the first connection region 16 and/or the second connection region 18, which are as described herein above.

FIG. 6A, FIG. 6B, FIG. 6C and FIG. 6D show a third embodiment of the guide. The third embodiment of the guide comprises many of the same features as the first embodiment and the second embodiment of the guide described above. The guide comprises a elongate body 10″, a slot 12, a second slot 12′, a third slot 12″ and a fourth slot 12′″. The elongate body 10″ comprises a first end 10″A and a second end 10″B and a longitudinal axis X extends therebetween. At least one difference between the first embodiment and the third embodiment is that the elongate body 10″ has multiple slots. The slots 12″ and 12′″ are all as described herein above in respect of the slot 12 and the second slot 12′. The third slot 12″ has a first end 12″A that is defined proximal the first end 10″A and a second end 12″B that is defined proximal to the second end 10″B. The fourth slot 12′″ has a first end 12′″A that is defined proximal the first end 10″A and a second end 12′″B that is defined proximal to the second end 10″B. The second end 12″B of the third slot 12″ is, similar to the slot 12, configured to define a desired angle relative to the longitudinal axis X at which the probe within the third slot 12″ may exit and extend away from the elongate body 10″. The second end 12′″B of the fourth slot 12′″ is, similar to the slot 12, configured to define a desired angle relative to the longitudinal axis X at which the probe within the fourth slot 12′″ may exit and extend away from the elongate body 10″. The desired angle defined by the second end 12″B and the second end 12′″B may be the same, or not, and they may be the same as the desired angle defined by the second 12B of the slot 12, or not.

The elongate body 10″ may also define the secondary slot 14 with the first end 14A defined proximal the first end 10″A and the second end 14B defined proximal the second end 10″B. As described above, the secondary slot 14 can be used to introduce another type of probe into a subject.

FIG. 6E shows an optional variation of the third embodiment that includes the first connection region 16 and/or the second connection region 18, which are as described herein above.

FIG. 7A, FIG. 7B, FIG. 7C and FIG. 7D show a fourth embodiment of the guide. The guide comprises a elongate body 20 that has a first end 20A and a second end 20B and a longitudinal axis X is defined therebetween. The elongate body 20 defines a slot 222 that is a coaxial with the longitudinal axis X of the elongate body 20. The slot 22 has many of the same features as the slot 12 described herein above. The slot 22 has a first end 22A defined proximal the first end 20A and a second end 22B defined proximal the second end 20B. The first end 22A of the slot 22 is configured to receive a probe therethrough. At least one difference between the slot 12 and the slot 22 is that the second end 22B of the slot 22 comprises a direction member 26 that is positioned outside of the slot 22. The direction member 26 is configured to define a desired angle relative to the longitudinal axis X at which the probe may exit and extend away from the elongate body 20. As shown in FIG. 7C, the direction member 26 may be radiussed so that as the probe exits the slot 22 the probe will bend and extend away from the elongate body 20 at the desired angle relative to the longitudinal axis X of the elongate body 20. In some embodiments of the present disclosure, the desired angle is between 5 and 90 degrees relative to the longitudinal axis X of the elongate body. In some embodiments of the present disclosure, the desired angle may be greater than 90 degrees relative to the longitudinal axis X of the elongate body 20. In some embodiments of the present disclosure, the predetermined angle can be modified by design of the direction member 26, by the selection of materials that the probe is constructed of, and/or the design of the probe itself.

The slot 22 may have a cross-sectional shape that corresponds to the cross-sectional shape of the probe. In some embodiments of the present disclosure, the slot 22 has a cross-sectional shape selected from one of a rectangular shape (as shown in FIG. 7), a square shape, a circular shape or a curvilinear shape.

In the fourth embodiment depicted in FIG. 7, the elongate body 20 also defines a secondary slot 24 that may be coaxial with the longitudinal axis X of the elongate body and the slot 22. The secondary slot 24 has a first end 24A that is defined proximal the first end 20A and a second end 24B that is defined proximal to the second end 20B. The first end 24A of the secondary slot 24 may be configured to receive another type of probe therein so that that the probe can move through the secondary slot 24 in a first direction from the first end 24A towards the second end 24B beyond the second end 24B the probe can continue moving in the first direction and extend away from the elongate body 20.

Although not shown in FIG. 7, the person skilled in the art will appreciate that the elongate body 20 may have, but does not require, the first connection region 16 and/or the second connection region 18.

The person skilled in the art will also readily appreciate that the specific descriptions of the embodiments hereinabove are illustrative only and not limiting. For example, the person skilled in the art will appreciate that embodiments of the guide may include as many slots as desired and limited only by the cross-sectional area of the elongate body. The person skilled in the art will also appreciate that the configuration of the second end of the slots may be internal (as in the radiussed portion of slot 12) or external (as in the direction member 26) or both. Furthermore, the person skilled in the art will appreciate that the slot(s) may or may not be defined by the elongate body.

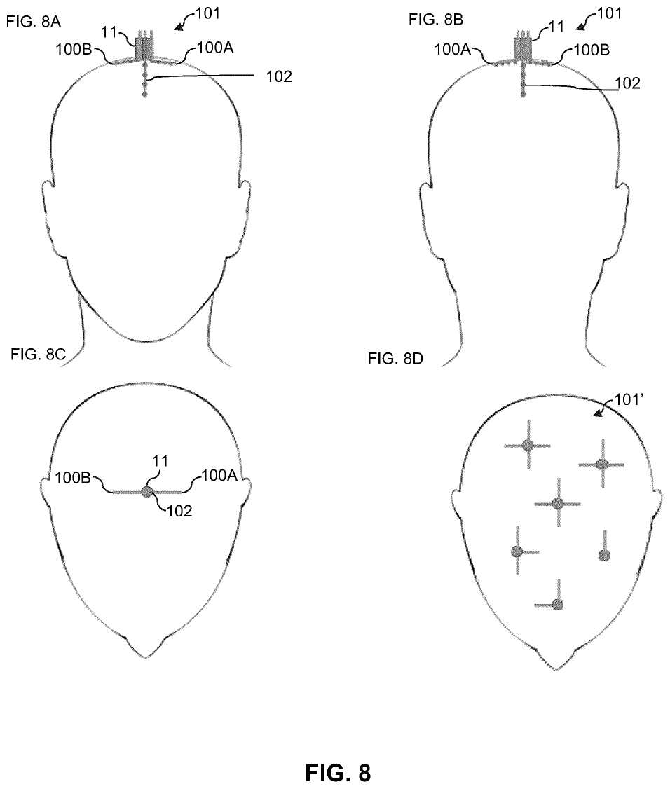

FIG. 8A, FIG. 8B, FIG. 8C and FIG. 8D each shows a different view of a system 101 that comprises a elongate body 11 that can be any one of the embodiments of the guide described herein above. The system 101 also comprises one neurocortical probe 100A and a second neurocortical probe 100B and a subcortical probe 102. As shown in FIG. 8A and FIG. 8B the neurocortical probe 100A and the neurocortical probe 100B both extend away from the elongate body 11 at a desired angle relative to the longitudinal axis X of the elongate body 11. FIG. 8D shows another example of the system 101′ that comprises multiple tubular bodies. Each elongate body is associated with one or more neurocortical probe and, optionally, a subcortical probe (not shown in the top plan view of FIG. 8D).

FIG. 9 shows non-limiting variations of the system 101 that include different embodiments of the guide that extends through a skin layer 200, a periosteum layer 202, a skull 204, a dura matter layer 206 of a subject and through the subarachnoid space 207 into the brain tissue 208, which includes both grey matter and white matter and neurocortical tissue 208A and subcortical tissue 208B. The probes 100, 100A and 100B are shown in FIG. 9 as being positioned upon or near a portion of the subject's neurocortical tissue 208A and the probe 102 is shown extending into the subject's subcortical tissue 208B. As will be appreciated by the person skilled in the art, the guide can be used to introduce one or more probes into other types of tissue within the subject, such as epithelial tissue, muscle tissue, connective tissue, nervous tissue—other than neurocortical tissue and subcortical tissue—or combinations thereof.

As will be appreciated by the person skilled in the art, because the guide may be inserted into the body of a subject, the guide may be constructed of a material that will reduce or avoid inducing an immune reaction by the subject. Examples of such materials include, but are not limited to: stainless steel, titanium, an alloyed metal, a coated metal where the coating reduces or avoids inducing an immune reaction and other materials and coatings that are or become known.

Some embodiments of the present disclosure relate to a method of introducing one or more probes into a subject's body. This method includes a step of setting up and preparing the subject and the probing site to be instrumented with the guide and probes. This step includes determining which types and numbers of probes or tools are going to be used, where the guide will be inserted (e.g. what is the target tissue: epithelial tissue, muscle tissue, connective tissue, nervous tissue or combinations thereof), how the guide will be inserted, what angle the probe should extend from the guide, what physiological data will be collected, and so on.

The method comprises a further step of accessing an interior of the subject's body via an access hole to introduce the one or more probes into, upon or near the target tissue; inserting the guide to the desired position. The method further comprises a step inserting the desired type and number of probes into the slots of the guide so that a second end of each probe extends from the elongate body at the desired angle for positioning the second end of each the probes near, upon, or inside the target tissue structure within the subject's body. Next, the positioning of the guide and probe(s) can be verified and the subject can be prepared for a step of acquiring the desired physiological data via the probe and transmitting that data to a processor and/or display unit. As a final step, the physiological data can be processed and interpreted to generate physiological information.

I claim:

1. A guide for inserting one or more probes into a subject, the guide comprising:
an elongate body with a first end and a second end, the elongate body defines one or more slots between the first end and the second end, each of the one or more slots are configured to receive a probe at the first end and to define a desired angle relative to the slot at which the probe is extendible from the second end.

2. The guide of claim 1, wherein the elongate body further defines a secondary slot that extends between the first and the second end, wherein the secondary slot is configured to receive a further probe that does not require an angular deflection upon extending from the second end of the elongate body.

3. The guide of claim 1, wherein the desired angle is between about 1 degree and about 179 degrees relative to a longitudinal axis of the elongate body.

4. The guide of claim 1 or claim 2, wherein the slot has a cross-sectional shape that is one of rectangular, square, circular or curvilinear.

5. The guide of claim 1 or claim 2, wherein the elongate body defines a portion of each of the one or more slots, the portion forms a directing member that is positioned proximal the second end, wherein the directing member is configured to define the desired angle relative to the slot at which the probe is extendible from the second end.

6. The guide of claim 1 or claim 2, wherein the elongate body further comprises a directing member that is proximal the second end, where in the directing member is configured to define the desired angle relative to the slot at which the neurocortical electrode is extendible from the second end.

7. The guide of claim 1 or claim 2, further comprising at least one further slot between the first end and the second end, wherein each of the at least one further slots are configured to receive a further probe at the first end and to define a desired relative to the slot at which the further probe is extendible from the second end.

8. The guide of claim 1 or claim 2, further comprising a sealing apparatus that is reversibly connectible to the first end, and wherein the sealing apparatus is configured to provide a fluid tight seal about the first end.

9. A system for acquiring a neurocortical recording, the system comprising:
(a) a guide for inserting a probe, the guide comprising: an elongate body with a first end and a second end, the elongate body defines a slot between the first end and the second end, the slot is configured to receive the probe at the first end and to define an angle relative to the slot at which the probe is extendible from the second end; and
(b) the probe, which is configured to acquire physiological data signals when positioned proximal to or upon a portion of a subject's neuro-cortex.

10. The system of claim 9, wherein the probe is further configured to deliver a therapy or a treatment.

11. The system of claim 9, wherein the elongate body further defines a secondary slot that extends between the first end and the second end, wherein the secondary slot is configured to receive a further probe that does not require an angular deflection upon extending from the second end of the elongate body.

12. The system of claim 9, wherein the further probe is a subcortical electrode.

13. The system of claim 9, wherein the further probe is a surgical tool.

14. The system of claim 8 wherein the further probe comprises a subcortical electrode and a surgical tool.

15. The system of claim 13 or claim 14, wherein the surgical tool is configured to deliver one or more therapies or treatments.

16. The system of claim 13 or claim 14, wherein the surgical tool is one or more of a sensor, an oxygen probe, a temperature probe, a chemically-sensitive probe, an optical fiber, a stent insertion assembly, a stent, a neuro-medical device insertion assembly, a neuro-medical device, and any other tools that may be used introduce and position objects within the subject.

17. The system of claim 9, further comprising a sealing apparatus that is reversibly connectible to the first end and wherein the sealing apparatus is configured to provide a fluid tight seal about the first end.

18. A method of introducing a probe into a subject's body, the method comprising steps of:
(a) accessing an interior of the subject's body via an access hole;
(b) inserting the guide of claim 1 or claim 2 into the hole so that the second end of the elongate body is proximal to a desired location within the subject's body; and
(c) inserting a probe into the slot so that a second end of the probe extends from the elongate body at the desired angle for positioning the second end of the probe near or upon a desired structure within the subject's body.

19. The method of claim 18, further comprising a step of acquiring desired physiological data from the desired structure.
